(19) United States
(12) Patent Application Publication
(10) Pub. No.: US 2026/0308268 A1
(43) Pub. Date: Oct. 8, 2026

(54) A PROIMMUNOTOXIN THERAPEUTIC NANOPLATFORM AGAINST CANCER

(71) Applicant: THE REGENTS OF THE UNIVERSITY OF CALIFORNIA, Oakland, CA (US)

(72) Inventors: Manuel L. PENICHET, Los Angeles, CA (US); Jing WEN, Los Angeles, CA (US); Tracy R. WELLS, Los Angeles, CA (US); Shilin CHEN, Los Angeles, CA (US)

(73) Assignee: THE REGENTS OF THE UNIVERSITY OF CALIFORNIA, Oakland, CA (US)

(21) Appl. No.: 19/479,189

(22) PCT Filed: May 1, 2024
(86) PCT No.: PCT/US2024/027293

§ 371 Date: Oct. 27, 2025

Related U.S. Application Data
(60) Provisional application No. 63/499,387, filed on May 1, 2023.

(51) Int. Cl. C07K 16/28; A61K 9/51; A61K 38/16; A61K 39/00; A61K 47/64; A61P 35/04

(52) U.S. Cl. CPC C07K 16/2881; A61K 9/5123; A61K 38/168; A61K 47/642; A61P 35/04; A61K 2039/505; C07K 2317/24; C07K 2317/55; C07K 2317/565; C07K 2317/622; C07K 2319/01

(57) ABSTRACT

Aspects of the disclosure relate to compositions comprising encapsulated immunotoxins and methods for using the encapsulated immunotoxins. The encapsulated immunotoxins may be capable of reaching otherwise difficult to target tissues, such as the brain, and may resist denaturation and degradation prior to reaching the targeted tissue.

This application claims priority of U.S. Provisional Application No. 63/499,387, filed May 1, 2023, which is hereby incorporated by reference in its entirety.

### STATEMENT OF GOVERNMENT SUPPORT

This invention was made with government support under CA253215, and CA196266 awarded by the National Institutes of Health. The government has certain rights in the invention.

### BACKGROUND

### 1. Field of the Invention

This invention relates to the field of molecular biology, genetic engineering, chemical engineering, and medicine.

### 2. Background

Non-Hodgkin lymphoma (NHL) is the most prevalent hematopoietic malignancy in the United States¹. Notably, individuals infected with the human immunodeficiency virus (HIV) are at an elevated risk for NHL²⁻⁵, along with an increased risk of both primary and metastatic brain involvement, leading to poorer survival rates⁶⁻⁸.

Transferrin receptor 1 (TfR1/CD71), a type II transmembrane homodimeric protein, is widely overexpressed on tumor cells, making it a universal cancer marker⁹⁻¹⁶. TfR1 can be targeted by antibodies for cancer therapy in two distinct ways: (1) directly, through antibodies that disrupt the function of the receptor and/or induce Fc effector functions, such as antibody-dependent cell-mediated cytotoxicity (ADCC), antibody-dependent cell-mediated phagocytosis (ADCP), or complement-dependent cytotoxicity (CDC), or (2) indirectly, through antibodies conjugated to antitumor agents that are internalized via receptor-mediated endocytosis 10-12,14,17,18. However, systemic delivery of anti-TfR1 antibodies, especially when conjugated to highly cytotoxic compounds, raises safety concerns due to the potential targeting of normal cells.

### SUMMARY OF THE INVENTION

In general, the disclosure relates to the discovery of toxins, which include immunotoxins (which include tumor selective targeting ligands such as antibodies linked to highly toxic protein molecules), encapsulated in a polymer shell, which may be referred to as proimmunotoxins. The polymer shell can be used to shield the toxin from denaturation or degradation.

Aspects of the disclosure relate to therapeutic agents, including nanodrugs, comprising a toxin, which can include an immunotoxin, encapsulated in a polymer shell. The disclosed immunotoxins include targeting molecules, such as antibodies, conjugated to a toxin. The antibody can be an antibody that targets a tumor-associated antigen. The antibody can be any antibody that targets TfR1, including ch128.1 and/or hu128.1. The antibody can be any type of antibody including a human antibody, a mouse antibody, a humanized antibody, or chimeric antibody. The chimeric antibody can be a mouse/human antibody, including ch128.1. Example of antibodies include those disclosed in U.S. patent application Ser. No. 17/775,167, PCT App. No. PCT/US2021/023735, and PCT App. No. PCT/US2021/048714, which are all hereby incorporated by reference for all purposes. Also disclosed are immunotoxins comprising a functional fragment of any antibody disclosed herein. Also disclosed are peptides and/or aptamers that bind to TfR1.

The toxin can be any cytotoxic agent, including any cytotoxic agent capable of killing a cancer cell. The toxin can be an artificial cytotoxic compound. The toxin can be a natural cytotoxic compound. The toxin can be any poisonous agent, produced by an organism. In certain aspects, the toxin is produced by bacteria. In certain aspects, the toxin is endogenously produced by bacteria. In certain aspects, the toxin is produced by an animal. In certain aspects, the toxin is endogenously produced by an animal. In certain aspects, the toxin is produced by a plant. In certain aspects, the toxin is endogenously produced by a plant. In certain aspects, the toxin is produced by a fungus. In certain aspects, the toxin is endogenously produced by a fungus. In some aspects, the toxin comprises a ribosome-inactivating protein (RIP), such as saporin. Also disclosed are immunotoxins comprising saporin, diphtheria toxin, Pseudomonas aeruginosa exotoxin A, ricin, or any combination thereof. Also disclosed are immunotoxins comprising monomethyl auristatin E (MMAE) or other antineoplastic agents that cannot be delivered systematically. The targeting molecule can be conjugated to the toxin by any means, such as biotin-avidin and/or biotin-streptavidin binding. Also disclosed are antibody-avidin fusion proteins. The targeting molecule can be conjugated to the toxin by covalent or non-covalent conjugation, including as a fusion protein or chemical crosslinking.

The toxin, which can include an immunotoxin, can be encapsulated in any suitable polymer shell, including a biodegradable polymer shell. The polymer shell can comprise 2-methacryloyloxyethyl phosphorylcholine (MPC). The polymer shell can be a degradable polymer shell. In certain aspects, the degradable polymer shell can be degraded by an enzyme. The degradable polymer shell may be degradable by cancer-specific or cancer-related enzymes. Cancer-related enzymes may include enzymes that are present in the tumor microenvironment (TME). Cancer-related enzymes may include enzymes that digest tissue, including tissue surrounding a tumor, and/or enzymes that promote cancer dissemination. Also disclosed are polymer shells degradable by enzymes present in the TME, including matrix metalloproteinase (MMP)-2, MMP-14, urokinase-type plasminogen activator (uPA), membrane type 1 serine protease (MT-SP1/matriptase), legumain, or any combination thereof. The polymer shell can comprise additional targeting molecules, which can be antibodies and/or ligands that interact and/or bind to cancer cells and/or tumor tissue. The additional targeting molecules can be chemically linked or otherwise incorporated into the polymer shell, including during polymerization of the polymer shell. The additional targeting molecules can comprise antitumor antigen antibodies, molecules capable of targeting brain tissue, molecule comprising blood-brain barrier (BBB) ligand, or any combination thereof. The additional targeting molecules can comprise chemokines, such as CXCL13. Also disclosed are polymer shells, which may include additional targeting molecules, such as the choline analog MPC to induce transcytosis across the BBB through choline transporters (ChTs), and thus, capable of targeting the central nervous system (CNS), including the brain.

The targeting molecules described herein can be conjugated to the toxins and/or polymer shells described herein using any conjugation method. The protein and toxin and/or polymer can be conjugated using fusion chemistry, such as click chemistry, which may be copper-free click chemistry.

Certain aspects relate to compositions comprising any of the encapsulated toxins, including any immunotoxin, described herein. The composition can also comprise other agents, including an additional therapeutic composition.

Also described herein are methods including but not limited to methods of treating a patient, or methods of delivering a toxin, including an immunotoxin, to a cancer cell, or methods of increasing localization of an immunotoxin to a cancer cell, or methods of increasing the effective amount of a toxin, including an immunotoxin, to a cancer cell in a patient, any of which methods can comprise the step of administering to the patient any of the encapsulated toxins, which may be referred to as proimmunotoxins and/or proimmunotoxin nanodrugs, described herein. In certain aspects, the patient has a disease, has been diagnosed with, has one or more symptoms of, or is suspected of having a disease, that is indicated for the encapsulated toxin. In certain aspects, the patient has, has been diagnosed with, has one or more symptoms of, or is suspected of having, a cancer, such as a brain cancer or a lymphoma. In certain aspects, the patient has non-Hodgkin lymphoma (NHL). In certain aspects, the cancer is NHL in the brain, which may be primary or metastatic. In certain aspects, the cancer is a solid tumor, which may be primary or metastatic. In certain aspects, the cancer comprises a brain tumor, which may be a cancer that did not originate in the brain or neuronal cells. In certain aspects, the cancer comprises cancerous cells in a deep tissue of the patient. In certain aspects, the cancer comprises one or more tumors in the patient, which can include surface tumors. In certain aspects, the cancer can be hematopoietic (i.e., blood cancer), including as NHL, or from epithelial or mesenchymal origin. The encapsulated immunotoxins can be delivered by any method, including subcutaneously, intravenously, intraperitoneally, intratumorally, or intrathecally. Also disclosed are methods where an additional therapy is administered to the patient.

### BRIEF DESCRIPTION OF THE DRAWINGS

The drawings form part of the present specification and are included to further demonstrate certain aspects of the present disclosure. The disclosure may be better understood by reference to one or more of these drawings in combination with the detailed description of specific embodiments presented herein.

FIG. 1 illustrates the design and action mechanism of the proimmunotoxin. The process begins with the formation of the immunotoxin ch128.1Av/b-SO6, where the anti-transferrin receptor 1 (TfR1) IgG3-avidin fusion protein (ch128.1Av) and the biotinylated saporin 6 (b-SO6) combine through avidin-biotin interaction. 2-methacryloyloxyethyl phosphorylcholine (MPC) monomer with choline analogues and peptide degradable crosslinkers are enriched around individual immunotoxin molecule (1). A thin layer of polymer shell gradually forms on the surface of the immunotoxin molecule through in situ polymerization of the monomers and crosslinkers, synthesizing the proimmunotoxin n(ch128.1Av/b-SO6) (2). Upon intravenous administration, the choline analogues on the proimmunotoxin facilitate its transport across the blood-brain barrier (BBB) via choline transporters (ChTs) (3). Within the tumor microenvironment (TME), the encapsulated immunotoxin is selectively released in response to proteolytic enzymes secreted by tumors, such as matrix metalloproteinase (MMP)-2 (4).

FIGS. 2a and 2b show characterization of proimmunotoxin to confirm its successful synthesis. Transmission electron microscopy (TEM) image showing the spherical morphology of the proimmunotoxin n(ch128.1Av/b-SO6), confirming its successful synthesis (FIG. 2a). The sizes of both native ch128.1Av/b-SO6 and n(ch128.1Av/b-SO6) were characterized using dynamic light scattering (DLS) (FIG. 2b).

FIG. 3 shows the biodistribution of native ch128.1Av/b-SO6 or n(ch128.1Av/b-SO6) in major organs of male BALB/c mice (week 6, n=3) monitored by IVIS fluorescence imaging 12 hours post-injection. 4 mg/kg of native ch128.1Av/b-SO6 or n(ch128.1Av/b-SO6) labeled with the fluorophore TAMRA was administered intravenously via tail vein. The representative biodistribution images showed improved delivery to the brain and lymph nodes and decreased accumulation to the liver, kidneys, and lung by n(ch128.1Av/b-SO6), compared to native ch128.1Av/b-SO6. The organ optical images and fluorescent images of labeled ch128.1Av/b-SO6 or n(ch128.1Av/b-SO6) were overlayed to visualize the immunotoxin or the proimmunotoxin within each organ. The dosage amount of ch128.1Av/b-SO6 and n(ch128.1Av/b-SO6) were normalized to the amount of ch128.1Av antibody fusion protein.

FIGS. 4a and 4b show the activity of n(ch128.1Av/b-SO6) using 2F7-BR44 spheroids in vitro. 2F7-BR44, a human AIDS-associated non-Hodgkin lymphoma (AIDS-NHL) clone genetically marked with mCherry reporter protein, were cultured in media containing 5% Matrigel gel for 5 days to form tumor spheroids. The killing activity of control phosphate-buffered saline (PBS), ch128.1Av/b-SO6, or n(ch128.1Av/b-SO6) at a concentration of 4.6 μg/mL was assessed by measuring the loss of mCherry expression in the spheroids after treatment. The assessment was conducted in triplicate. Representative fluorescent images of 2F7-BR44 spheroids treated with PBS, ch128.1Av/b-SO6, or n(ch128.1Av/b-SO6) for 24 and 48 hours, showing cell viability (FIG. 4a). White circles indicate areas of cell loss due to n(ch128.1Av/b-SO6) treatment. The sizes of spheroids were measured and compared among treatments, indicating the inhibited growth of the outer layers of spheroids by treatment (FIG. 4b). Data are presented as means±standard deviation (S.D.), with significance calculated using the Student's t-test with Welch's correction. ****p<0.0001.

FIGS. 5a and 5b show the penetration capability of n(ch128.1Av/b-SO6) explored using a Hoechst dye staining assay. 2F7-BR44 spheroids were prepared using the same protocol as FIG. 4 and stained with 0.1 μM Hoechst dye. Spheroids were dissociated into single cell suspension and cells displaying dye intensity lower than 10⁴ were identified as the core via flow cytometry (FIG. 5a). Cell viability was assessed by mCherry expression. Tumor penetration efficacy of ch128.1Av/b-SO6 and n(ch128.1Av/b-SO6) was compared based on cell viability within the core of spheroids (FIG. 5b). Data are presented as means±S.D. Significance was calculated with Student's t-test with Welch's correction between ch128.1Av/b-SO6 and n(ch128.1Av/b-SO6) groups. ns: not significant. *p<0.05.

FIGS. 6a-6c show that the proimmunotoxin n(ch128.1Av/b-SO6) can effectively shield the toxicity of encapsulated immunotoxin. Male NSG mice aged 6 weeks received 4 mg/kg of ch128.1Av/b-SO6 or n(ch128.1Av/b-SO6) via intravenous injection administered retro-orbitally (n=3). The dosage amount of ch128.1Av/b-SO6 and n(ch128.1Av/b-SO6) were normalized to the amount of ch128.1Av antibody fusion protein. Kaplan-Meier survival plots of mice were plotted relative to the numbers of days after injection (FIG. 6a). Significance was calculated with log-rank test. *p<0.05. Comparison of plasma levels of alanine aminotransferase (ALT), an indicator of acute liver toxicity, between treatments using ch128.1Av/b-SO6 or n(ch128.1Av/b-SO6). Data are presented as means±S.D. (FIG. 6b). Representative photographs depicting the eye conditions of NSG mice at day 1 post-injection, indicating reduced eye inflammation in mice treated with n(ch128.1Av/b-SO6) compared to those treated with ch128.1Av/b-SO6. (FIG. 6c).

FIGS. 7a and 7b show the therapeutic efficacy of n(ch128.1Av/b-SO6) in a xenograft AIDS-NHL model using 2F7-BR44 genetically marked with luciferase for imaging purpose. Male NSG mice aged 6 weeks were xenografted with 10⁶ 2F7-BR44 intravenously via tail vein to establish disseminated lymphoma with brain metastases. Longitudinal IVIS bioluminescent imaging of tumor progression in NSG mice xenografted with 2F7-BR44 and treated with 0.1 mg/kg of ch128.1Av/b-SO6, n(ch128.1Av/b-SO6), or control PBS intravenously via tail vein, on days 7 and 21 post-xenograft (control n=3, treatment n=4) (FIG. 7a). Boxes containing a red X indicate deceased mice. Quantitative analysis of tumor burden was quantified and compared among treatment as the total bioluminescent intensity (BLI) per second within a region of interest (whole body or head area), based on IVIS imaging (FIG. 7b). The dosage amount of ch128.1Av/b-SO6 and n(ch128.1Av/b-SO6) were normalized to the amount of ch128.1Av antibody fusion protein. Data are presented as means±S.D. Significance was calculated with Student's t-test with Welch's correction between ch128.1Av/b-SO6 and n(ch128.1Av/b-SO6) groups. ns: not significant, *p<0.05, **p<0.01.

FIG. 8 shows the analysis of the tumor cell percentages in the kidneys and bone marrow (BM) of the AIDS-NHL xenograft mice in the therapeutic study shown in FIG. 7. Single cells isolated from these organs were evaluated for mCherry+ cells via flow cytometry to monitor tumor presence. Data are presented as means±S.D. Significance was calculated with Student's t-test with Welch's correction. ns: not significant, *p<0.05, ***p<0.001.

FIGS. 9a and 9b show the concentrations of native ch128.1Av/b-SO6 and released immunotoxin from n(ch128.1Av/b-SO6) in the organs of the AIDS-NHL xenograft mice in the therapeutic study shown in FIG. 7. Tissues were homogenized and supernatant were collected for the ELISA tests. The concentrations of native and released immunotoxin in organs bearing tumors, such as the kidneys and brain. Comparisons of immunotoxin levels between ch128.1Av/b-SO6 and n(ch128.1Av/b-SO6) groups highlight differences in biodistribution (FIG. 9a). Additional ELISA tests evaluated the accumulation of native and released immunotoxin in the spleen and liver, which are organs without tumor burden (FIG. 9b). This analysis helps assess unintended immunotoxin retention. Data are presented as means±S.D. Significance was calculated with Student's t-test with Welch's correction. ns: not significant, *p<0.05, ***p<0.001.

FIGS. 10a-10c show the further improved therapeutic efficacy of proimmunotoxin using CXCL13 ligands conjugation. CXCL13 is a chemokine associated with B-cell NHL and binds to CXCR5, a receptor expressed on B-cell NHL, to better target tumors. Male NSG mice aged 6 weeks were xenografted with 10⁶ 2F7-BR44 intravenously via tail vein to establish disseminated lymphoma with brain metastases. Longitudinal monitoring of tumor burden was assessed weekly through IVIS imaging in xenografted mice administered intravenously via tail vein with 1 mg/kg of PBS control, ch128.1Av/b-SO6, n(ch128.1Av/b-SO6), or n(ch128.1Av/b-SO6)^{CXCL13} (n=6). Boxes with a red X indicate deceased mice, illustrating treatment impacts on survival (FIG. 10a). Kaplan-Meier survival plots of AIDS-NHL xenografted mice treated with four treatments, highlighting the potential therapeutic benefits of CXCL13 ligand conjugation with the proimmunotoxin (FIG. 10b). The dosage amount of ch128.1Av/b-SO6, n(ch128.1Av/b-SO6), and n(ch128.1Av/b-SO6)^{CXCL13} were normalized to the amount of ch128.1Av antibody fusion protein. The median survival and the p-values (log-rank test) among different groups were summarized in the table (FIG. 10c).

### DESCRIPTION OF ILLUSTRATIVE ASPECTS

### I. General Aspects

The transferrin receptor 1 (TfR1), also known as CD71, has been identified as a universal cancer marker due to its overexpression on cancer cells, ability to internalize, and central role in cancer pathology⁹⁻¹⁶. This makes it an attractive target for cancer therapy using anti-TfR1 antibodies. TfR1 can be targeted by antibodies for cancer therapy in two distinct ways: (1) directly, through antibodies that disrupt the function of the receptor and/or induce Fc effector functions, such as antibody-dependent cell-mediated cytotoxicity (ADCC), antibody-dependent cell-mediated phagocytosis (ADCP), or complement-dependent cytotoxicity (CDC), or (2) indirectly, through antibodies conjugated to antitumor agents that are internalized via receptor-mediated endocytosis 10-12,14,17,18. However, systemic delivery of anti-TfR1 antibodies, especially when conjugated to highly cytotoxic compounds, raises safety concerns due to the potential targeting of normal cells.

Aspects herein relate to a high affinity antibody targeting human TfR1 genetically fused to avidin, to be used as a universal vector to deliver a broad spectrum of biotinylated agents into cancer cells by receptor-mediated endocytosis¹⁹. This antibody fusion protein, known as ch128.1Av (previously named anti-hTfR IgG3-Av), can eliminate the need of developing specific antibody fusion proteins or conjugates for each application and takes advantage of the natural high affinity of avidin for biotin, The conjugation of ch128.1Av with biotinylated saporin 6 (b-SO6), a plant RIP, can result in a potent immunotoxin (ch128.1Av/b-SO6), capable of eliminating different types of malignant B cells^{20,21}. In order to address the concerns on systemic toxicity in normal cells triggered by the presence of the toxin, the immunotoxin can be encapsulated with a polymer shell to form a proimmunotoxin nanodrug^{22,23}. The proimmunotoxin nanodrug can be formulated to unmask in the TME. The unmasking can be induced by enzymes expressed in the TME, such as a matrix metalloproteinase. The polymer shell can be synthesized with monomers and peptide-based crosslinkers through in situ polymerization^{24,25}. The specific cell killing of this proimmunotoxin nanodrug can be further improved by modifying it with cell type or tissue-specific ligands on the polymer shell. The proimmunotoxin nanodrug can shield the encapsulated immunotoxins from the environment and confer high resistance to denaturation, proteases, and nucleases. It can be inert, highly stable, resistant to protein adsorption, negligibly immunogenic, and/or biodegradable. The proimmunotoxin can reduce or ameliorate systemic toxicity, liver toxicity, and/or renal toxicity. The proimmunotoxin can provide effective antitumor efficacy by delivering and releasing immunotoxins only in the TME.

Certain aspects make possible the use of a highly toxic compound as a viable drug in vivo. The proimmunotoxin can comprise an antibody targeting TfR1 conjugated with the RIP toxin saporin (immunotoxin) inside a nanocapsule. However, other aspects relate to other antibodies and derivatives or targeting molecules specific for other tumor antigens, which are conjugated to saporin or other cytotoxic compounds. Other aspects can apply different types of nanocapsules or similar devices using the same principle. In addition, the nanocapsule can be “coated” with tumor targeting molecules including antibodies and ligands.

Disclosed herein are anti-TfR1 IgG3-avidin fusion protein named ch128.1Av, which can be used to deliver biotinylated therapeutic agents into cancer cells by receptor-mediated endocytosis. Conjugation of ch128.1Av with b-SO6, results in a potent immunotoxin (ch128.1Av/b-SO6) designed to eliminate malignant cells, including NHL malignancies; however, safe systemic delivery of ch128.1Av/b-SO6 can be limited by its toxicity to normal cells.

To prevent this problem and also allow BBB penetration, disclosed are proimmunotoxin nanodrugs, including n(ch128.1Av/b-SO6), which utilizes a nanoplatform. The immunotoxin can be encapsulated within a thin polymer shell formed by in situ polymerization of monomers with choline analogues. The nanocapsule can be stabilized with peptide crosslinkers responding to the TME-specific enzyme metalloproteinase-2 (MMP-2). In this strategy, the immunotoxin can be released intratumorally. The n(ch128.1Av/b-SO6) nanodrug can significantly eliminate non-tumor toxicity in vitro and in vivo, can exhibit increased penetration in the in vitro BBB model as well as the NHL spheroid model compared to native immunotoxin.

The proimmunotoxin n(ch128.1Av/b-SO6) conjugated with CXCL13, a chemokine associated with B-cell NHL that expresses the chemokine (CXCL13) receptor CXCR5, can specifically target tumors and ameliorate non-tumor toxicity in vitro and in vivo.

Overall, n(ch128.1Av/b-SO6)^{CXCL13}, and to a lesser extent n(ch128Av/b-SO6), demonstrates significant antitumor activity in an AIDS-NHL xenograft mouse model with brain metastases. This model based on the 2F7 cell line variant 2F7-BR44, which is more aggressive than the parental cell line (2F7) and forms metastases in the mouse brain after systemic (intravenous) administration²³. This unique proimmunotoxin nanodrug may provide an adequate therapeutic window, making it a relevant therapeutic for NHL and potentially other malignancies.

The proimmunotoxin platform is designed to minimize the side effects of a native immunotoxin, including ch128.1Av/b-SO6, and/or improve its ability to target the TME and penetrate the BBB.

The proimmunotoxin nanodrugs, including n(ch128.1Av/b-SO6), can enhance the therapeutic efficacy with prolonged half-life and minimized toxicity compared to native immunotoxin counterparts.

### II. Definitions

The terms “protein,” “polypeptide,” and “peptide” are used interchangeably herein when referring to a gene product.

The term “antigen-binding protein” refers to a protein that specifically binds to a certain antigen. In some aspects, the antigen-binding protein has no, undetectable, or minimal non-specific binding.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the N-terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In some embodiments, the methods are useful for reducing the size and/or cell number of a tumor. In some embodiments, the method of the disclosure are useful for inhibiting the growth of tumors, such as solid tumors, in a subject.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The antibody may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The term functional antibody fragment includes antigen binding fragments of antibodies, including e.g., Fab′, F(ab′)₂, Fab, Fv, rlgG, and scFv fragments. The term scFv refers to a single chain Fv antibody in which the variable domains of the heavy chain and of the light chain of a traditional two chain antibody have been joined to form one chain. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment retains the ability to bind its cognate antigen at comparable affinity to the full antibody.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include several different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical composition” or “pharmacologically acceptable composition” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition may be adjusted according to well-known parameters.

The term “unit dose” or “dosage” refers to physically discrete units suitable for use in a subject, each unit containing a predetermined quantity of the therapeutic composition calculated to produce the desired responses discussed herein in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any particular dose derivable therein. In non-limiting examples of a range derivable from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The use of a single chain variable fragment (scFv) is of particular interest. scFvs are recombinant molecules in which the variable regions of light and heavy immunoglobulin chains encoding antigen-binding domains are engineered into a single polypeptide. Generally, the V_{H} and V_{L} sequences are joined by a linker sequence. See, for example, Ahmad (2012) Clinical and Developmental Immunology Article ID 980250, herein specifically incorporated by reference. Described herein are BCMA-specific scFv molecules that comprise the variable regions of light and heavy immunoglobulin chains encoding BCMA-binding domains that are engineered into a single polypeptide. Similarly, the CS1-specific scFv molecules described herein comprise the variable regions of light and heavy immunoglobulin chains encoding CS1-binding domains that are engineered into a single polypeptide.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

Subject” and “patient” refer to either a human or non-human, such as primates, mammals, and vertebrates. In particular embodiments, the subject is a human.

Throughout this application, the term “about” is used to indicate that a value includes the inherent variation of error for the device, the method being employed to determine the value, or the variation that exists among the study subjects.

### III. Antigen-Binding Protein Conjugates

Aspects of the disclosure relate to antigen-binding proteins conjugated to an immunotoxin. The antigen-binding protein can be conjugated to the immunotoxin by any means, including covalently, ionically, and/or through protein-protein interactions. In some embodiments, the antigen-binding protein comprises an anti-TfR1 antibody or a fragment thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Rockberg and Nilvebrant (Eds.), Epitope Mapping Protocols, Humana Press, New York, NY, USA (2018). Such techniques are known in the art and described in, e.g., U.S. Pat. No. 4,708,871; Geysen et al., Proc. Natl. Acad. Sci., USA 81 (13): 3998-4002 (1984); Geysen et al., Proc. Natl. Acad. Sci., USA 82:178-182 (1985); and Geysen et al., Mol. Immunol., 23 (7): 709-715 (1986), each of which is incorporated by reference herein in their entirety. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front. Immunol., 4: Article 302 (2013))

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgAQ1 and IgA2.

Antibodies can be whole immunoglobulins of any isotype or classification, chimeric antibodies, or hybrid antibodies with specificity to two or more antigens. They may also be fragments (e.g., F(ab′) 2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79 (3): 315-321 (1990); Kostelny et al., J. Immunol. 148 (5): 1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA, 90 (14): 6444-6448 (1993); Polijak et al., Structure, 2 (12): 1121-1123 (1994); Todorovska et al., J. Immunol. Methods, 248 (1-2): 47-66 (2001), each of which is incorporated herein by reference in their entirety).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in Wu and Kabat, J. Exp. Med., 132 (2): 211-250 (1970)); Chothia (as described in Chothia et al., Nature, 342 (6252): 877-883 (1989)); and IMGT (as described in Lefranc et al., Dev. Comp. Immunol., 27 (1): 55-77 (2003)). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Biotechnology, 10 (7): 779-783 (1992) describes affinity maturation by V_{H} and V_{L} domain shuffling, random mutagenesis of CDR and/or FRs employed in phage display is described by Rajpal et al., Proc. Natl. Acad. Sci. USA, 102 (24): 8466-8471 (2005) and Thie et al., Methods Mol Biol., 525:309-322 (2009) in conjugation with computation methods as demonstrated in Tiller et al., Front. Immunol., 8: Article 986 (2017), each of which references are incorporated herein by reference in their entirety.

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. For methods relating to chimeric antibodies, see, e.g., U.S. Pat. No. 4,816,567; and Morrison et al., Proc. Natl. Acad. Sci. USA, 81 (21): 6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference in their entirety.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature, 321:522-525 (1986); Riechmann et al., Nature, 332:323-327 (1988); Presta, Curr. Op. Struct. Biol., 2:593-596 (1992); Vaswani and Hamilton, Ann. Allergy, Asthma and Immunol., 81 (2): 105-115 (1998); Harris, Biochem. Soc. Trans., 23 (4); 1035-1038 (1995); Hurle and Gross, Curr. Op. Biotech., 5:428-433 (1994); Verhoeyen et al., Science, 239 (4847): 1534-1536 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### 1. Functional Antibody Fragments and Antigen-Binding Fragments

a. Antigen-Binding Fragments

Certain aspects relate to antibody fragments, such as antibody fragments comprise the antigen-binding proteins described herein. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CHI) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Holt et al., Trends Biotechnol., 21 (11): 484-490 (2003)) constituted with a single VH or VL domain; (v) isolated complementarity determining region (CDR) regions. Such terms are described, for example, in Harlow and Lane (Eds.), Antibodies: A Laboratory Manual, Cold Spring Harbor Laboratory, New York, NY, USA (1988); Molecular Biology and Biotechnology: A Comprehensive Desk Reference, Meyers (Ed.), Wiley-VCH Publisher, Inc., New York, NY, USA (1995); Huston et al., Cell Biophys., 22 (1-3): 189-224 (1993); and Pluckthun and Skerra, Methods Enzymol., 178:497-515 (1989), each of which are incorporated by reference in their entirety.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′) 2 fragment means a bivalent antigen-binding fragment of a monoclonal antibody comprising two Fab′ fragments linked by a disulfide bridge at the hinge region. An F(ab′) 2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv) 2 means bivalent or bispecific sFv polypeptide chains that include oligomerization domains at their C-termini, separated from the sFv by a hinge region. The oligomerization domain comprises self-associating a-helices, e.g., leucine zippers, which can be further stabilized by additional disulfide bonds. (scFv) 2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

b. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included. The Fc region may be removed from an antibody to generate the antigen-binding proteins described herein. Removing the Fc region may allow for the antigen-binding protein to not induce an immune response against the antigen, or cell expressing the antigen, the antigen-binding protein targets.

c. Polypeptides with Antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13 (4): 167-187 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### IV. Cells

Certain embodiments relate to cells comprising polypeptides or nucleic acids of the disclosure, such as any of the antigen-binding proteins described herein. In some embodiments the cell is an immune cell or a T cell. “T cell” includes all types of immune cells expressing CD3 including T-helper cells, cytotoxic T-cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection® (ATCC®) No. CCL-2), CHO cells (e.g., ATCC® Nos. CRL9618, CCL61, CRL9096), human embryonic kidney (HEK) 293 cells (e.g., ATCC® No. CRL-1573), Vero cells, NIH 3T3 cells (e.g., ATCC® No. CRL-1658), Huh-7 cells, BHK cells (e.g., ATCC® No. CCL10), PC12 cells (ATCC No. CRL1721), COS cells, COS-7 cells (ATCC® No. CRL1651), RATI cells, mouse L cells (ATCC® No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell or progenitor cell obtained from an individual. In some embodiments, the cell used in therapy of a patient is autologous. In some embodiments, the cell used in therapy of a patient is non-autologous.

### V. Methods for Modifying Genomic DNA

In certain embodiments, the genomic DNA is modified either to include additional mutations, insertions, or deletions, or to integrate certain molecular contructs of the disclosure so that the constructs are expressed from the genomic DNA. In some embodiments, a nucleic acid encoding a polypeptide of the disclosure is integrated into the genomic DNA of a cell. In some embodiments, the integration is targeted integration. In some embodiments, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T-cell receptor alpha constant) locus. For instance, cells would first be electroporated with a ribonucleoprotein (RNP) complex consisting of Cas9 protein complexed with a single-guide RNA (sgRNA) targeting the TRAC (T-cell receptor alpha constant) locus. Fifteen minutes post electroporation, the cells would be treated with AAV6 carrying the HDR template that encodes for the CAR.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, ΦC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRIPSR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Multiple recognition sequences may be present in a single landing pad, allowing the landing pad to be targeted sequentially by two or more polynucleotide modification enzymes such that two or more unique nucleic acids (comprising, among other things, receptor genes and/or inducible reporters) can be inserted. Alternatively, the presence of multiple recognition sequences in the landing pad, allows multiple copies of the same nucleic acid to be inserted into the landing pad. When two nucleic acids are targeted to a single landing pad, the landing pad includes a first recognition sequence for a first polynucleotide modification enzyme (such as a first ZFN pair), and a second recognition sequence for a second polynucleotide modification enzyme (such as a second ZFN pair). Alternatively, or additionally, individual landing pads comprising one or more recognition sequences may be integrated at multiple locations. Increased protein expression may be observed in cells transformed with multiple copies of a payload Alternatively, multiple gene products may be expressed simultaneously when multiple unique nucleic acid sequences comprising different expression cassettes are inserted, whether in the same or a different landing pad. Regardless of the number and type of nucleic acid, when the targeting endonuclease is a ZFN, exemplary ZFN pairs include hSIRT, hRSK4, and hAAVS1, with accompanying recognition sequences.

Generally speaking, a landing pad used to facilitate targeted integration may comprise at least one recognition sequence. For example, a landing pad may comprise at least one, at least two, at least three, at least four, at least five, at least six, at least seven, at least eight, at least nine, or at least ten or more recognition sequences. In embodiments comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable or screenable genes as described herein, such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31 (11): 2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols, 7 (1): 171-192; Bogdanove A J, Voytas D F., 2011, Science, 333 (6051): 1843-6; Bradley P, Bogdanove A J, Stoddard B L., 2013, Curr. Opin. Struct. Biol., 23 (1): 93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some embodiments, targeted integrated can be achieved through the use of an integrase. For example, The phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been show to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one embodiment, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals. DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tcl/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

### I. Methods of Treatment

Aspects of the current disclosure relate to methods of treating a patient. The patient may be a cancer patient. The patient may be diagnosed with or suspected of having cancer. The methods generally involve administering an immunotoxin encapsulated in a polymer shell.

In some embodiments, the methods relate to administration of the antigen-binding protein in combination with a treatment for cancer (or other disease treated with a cytotoxic agent) or administration to a person with a cancer (or other disease treated with a cytotoxic agent).

Administration of the compositions according to the current disclosure will typically be via any common route. This includes, but is not limited to parenteral, orthotopic, intradermal, subcutaneous, intramuscular, intraperitoneal, or intravenous injection.

Typically, compositions of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 2, 3, 4, 5, 6, 7, 8, 9, 10 or more. The administrations may range from 2-day to 12-month intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### VI. Administration of Therapeutic Compositions

Methods of the disclosure include administration of a combination of therapeutic agents and/or administration of therapeutic agents, such as the encapsulated, for example. The therapy may be administered in any suitable manner known in the art. For example, the therapies may be administered sequentially (at different times) or concurrently (at the same time). In some embodiments, the therapies are in a separate composition. In some embodiments, the therapies are in the same composition.

Various combinations of the therapies may be employed, for example, one therapy designated “A” and another therapy designated “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies of the disclosure may be administered by the same route of administration or by different routes of administration. In some embodiments, the therapy is administered intracolonically, intravenously, intramuscularly, subcutaneously, topically, orally, transdermally, intraperitoneally, intraorbitally, by implantation, by inhalation, intrathecally, intraventricularly, or intranasally. In some embodiments, the microbial modulator is administered intravenously, intramuscularly, subcutaneously, topically, orally, transdermally, intraperitoneally, intraorbitally, by implantation, by inhalation, intrathecally, intraventricularly, or intranasally.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of a therapeutic composition that is administered to a human will be in the range of about 0.01 to about 50 mg/kg of patient body weight whether by one or more administrations. In some embodiments, the therapeutic agent used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the therapeutic agent is administered at 15 mg/kg. However, other dosage regimens may be useful. In one embodiment, a therapeutic agent described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Synthesis of Proimmunotoxin

### Methods

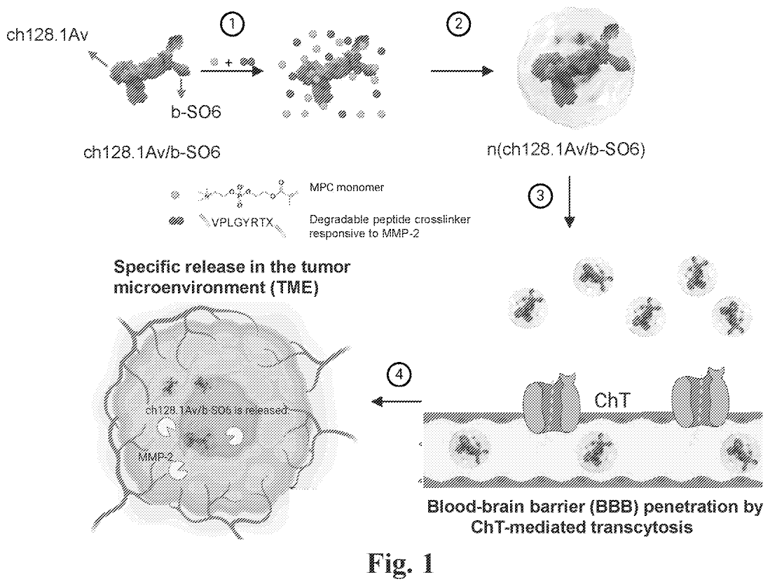

The anti-transferrin receptor (TfR1) IgG3-avidin fusion protein (ch128.1Av), produced in-house, was incubated with biotinylated saporin 6 (b-SO6) on ice for 30 minutes to form the immunotoxin ch128.1Av/b-SO6. A custom peptide crosslinker with the sequence VPLGVRTK (Biomatik, Wilmington, DE, USA) was acryloylated by reacting the amine groups on the valine and lysine residues with acrylic acid N-hydroxysuccinimide ester (NAS) (Sigma-Aldrich, St. Louis, MO, USA) at room temperature for one hour. This modified peptide was then dialyzed overnight against deionized water, lyophilized, and stored at −20° C. A mixture containing 2-methacryloyloxyethyl phosphorylcholine (MPC), N-(3-aminopropyl) methacrylamide (APM) (Sigma-Aldrich), and the acryloylated peptide crosslinkers at a molar ratio of 120:1:10 was prepared as a precursor stock solution. The proimmunotoxin n(ch128.1Av/b-SO6) was synthesized by encapsulating the immunotoxin in a polymer shell through free radical polymerization (FIG. 1). This process was initiated by adding the precursor stock solution (MPC/immunotoxin, 12000:1, n/n), ammonium persulfate (APS/immunotoxin, 500:1, n/n), and tetramethylethylenediamine (TEMED/APS, 2:1, w/w) to the ch128.1Av/b-SO6 solution and keeping it on ice for two hours. Finally, n(ch128.1Av/b-SO6) was dialyzed against phosphate-buffered saline (PBS) at 4° C. overnight and purified using a hydrophobic interaction column (Phenyl-Sepharose CL-4B, Sigma-Aldrich).

### Results

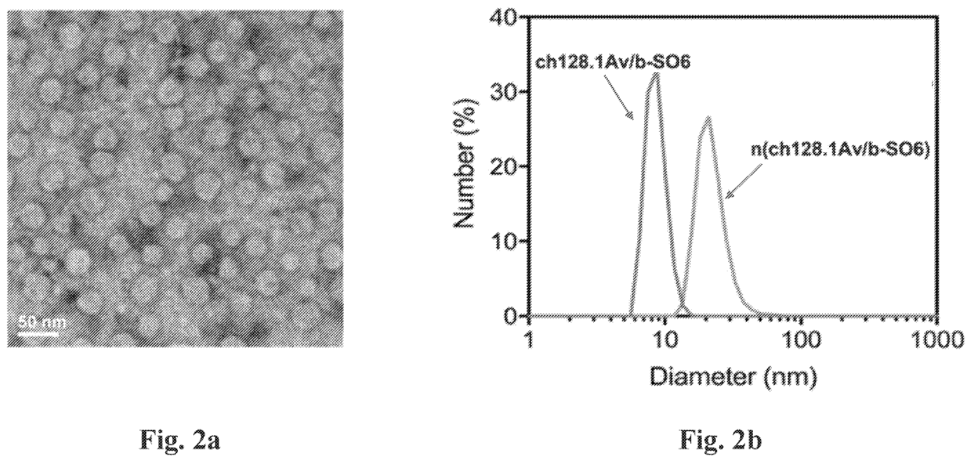

The successful synthesis of the proimmunotoxin n(ch128.1Av/b-SO6) was confirmed by transmission electron microscopy (TEM) and dynamic light scattering (DLS). FIGS. 2a-b show the TEM image of n(ch128.1Av/b-SO6) and the particle diameter distribution of both ch128.1Av/b-SO6 and n(ch128.1Av/b-SO6), respectively, measured using DLS. This analysis revealed spherical morphologies and uniformly larger particle sizes for n(ch128.1Av/b-SO6) compared to ch128.1Av/b-SO6, indicating the successful formation of a polymer shell around each molecule.

### Example 2—Biodistribution of Proimmunotoxin

### Methods

The organ distribution of ch128.1Av/b-SO6 and n(ch128.1Av/b-SO6) was assessed in male BALB/c mice aged week 6. ch128.1Av/b-SO6 was labeled with the fluorophore TAMRA for the synthesis of n(ch128.1Av/b-SO6). The dosages of both ch128.1Av/b-SO6 and n(ch128.1Av/b-SO6) were normalized based on the amount of ch128.1Av antibody in each sample. Mice were administered intravenously via tail vein with 4 mg/kg of either ch128.1Av/b-SO6 or n(ch128.1Av/b-SO6) (n=3). After 12 hours, the mice were euthanized, perfused, and their major organs were harvested for fluorescent imaging using the IVIS Lumina II In Vivo Imaging System (PerkinElmer, Waltham, MA).

### Results

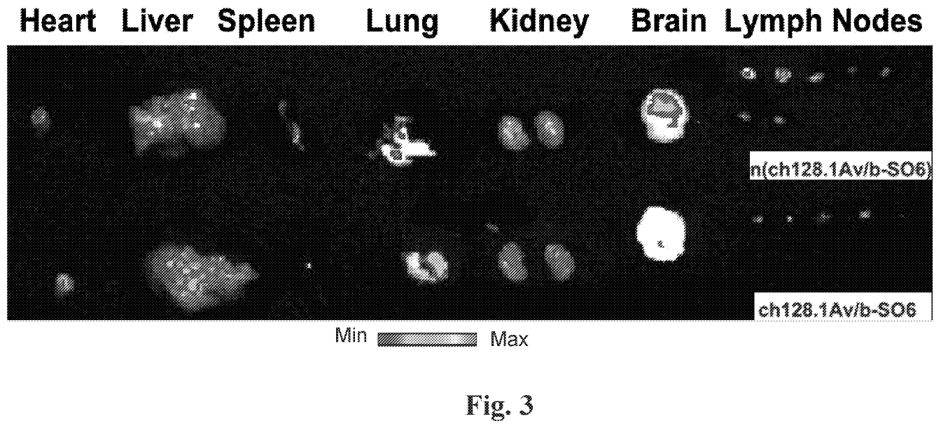

FIG. 3 shows the representative fluorescent images of major organs from mice treated with either ch128.1Av/b-SO6 or n(ch128.1Av/b-SO6). Compared to ch128.1Av/b-SO6, n(ch128.1Av/b-SO6) shows enhanced delivery to sanctuary sites, such as the brain. In addition, undesired accumulation in the liver, lungs, and kidneys is reduced by n(ch128.1Av/b-SO6). These findings suggest that the proimmunotoxin has the potential to reduce the side effects associated with the native immunotoxin.

### Example 3—In Vitro Tumor Killing and Penetration Assay with 3D Tumor Spheroids

### Methods

The ability of ch128.1Av/b-SO6 and n(ch128.1Av/b-SO6) to kill tumor cells and penetrate deeply into solid tumors was assessed using 3D tumor spheroids formed with 2F7-BR44 cells. The 2F7-BR44, a human AIDS-associated non-Hodgkin lymphoma (AIDS-NHL) clone genetically marked with an mCherry reporter protein, was cultured in media containing 5% Matrigel for 5 days to form tumor spheroids. A concentration of 4.6 μg/mL of ch128.1Av/b-SO6 or n(ch128.1Av/b-SO6) was added to the culture media of the spheroids. Fluorescent images of 2F7-BR44 spheroids treated with control phosphate-buffered saline (PBS), ch128.1Av/b-SO6, or n(ch128.1Av/b-SO6) were taken at 24 and 48 hours to evaluate cell viability by monitoring mCherry expression. The assessment was conducted in triplicate. The sizes of spheroids under different treatments were measured using ImageJ software. The penetration capability of ch128.1Av/b-SO6 and n(ch128.1Av/b-SO6) into 3D tumor spheroids was further examined using a Hoechst dye staining assay, where 0.1 μM Hoechst dye was added to the spheroid culture media and incubated for 15 minutes, enabling differentiation of the core and outer layer of the tumor spheroids based on the staining intensity at 10⁴ relative fluorescent units (RFU). The 3D tumor spheroids were dissociated, and both Hoechst intensity and mCherry expression in each cell was analyzed using a BD LSRFortessa flow cytometer (BD Biosciences, San Jose, CA, USA).

### Results

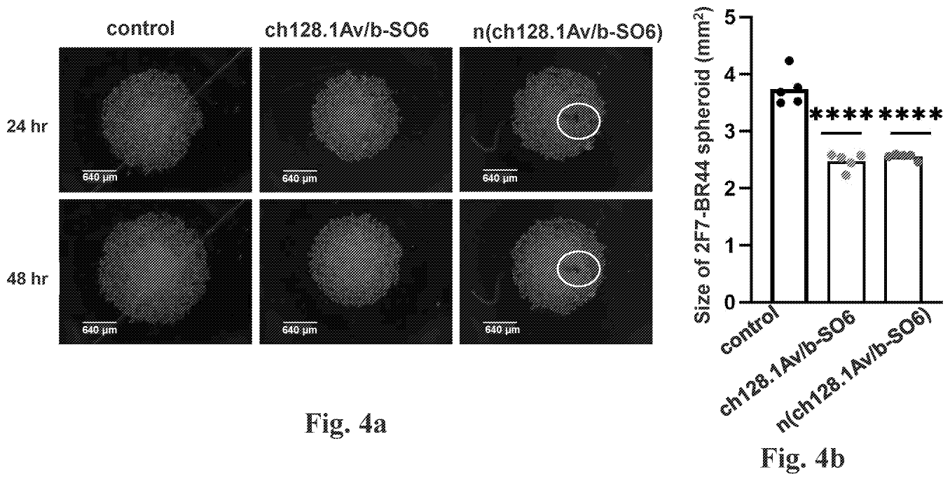

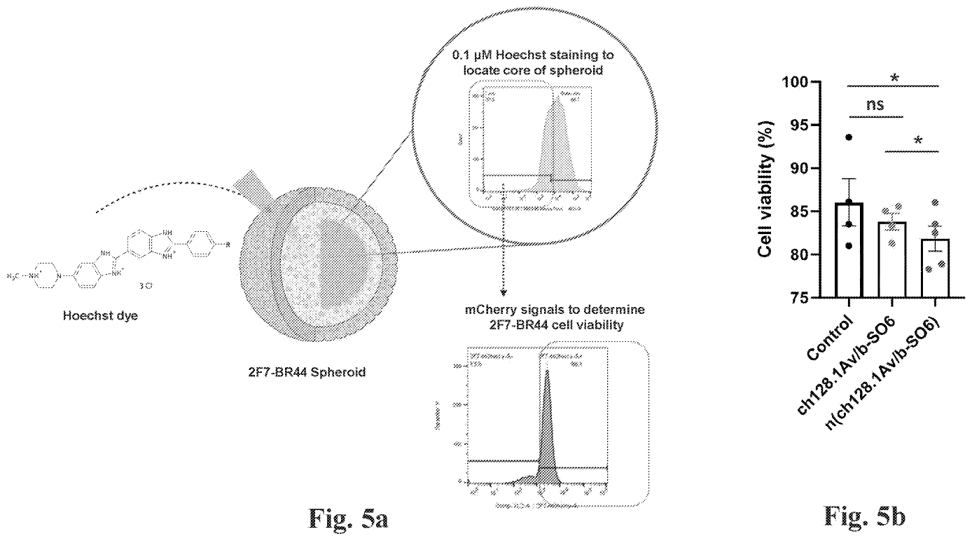

FIGS. 4a-b and 5a-b demonstrate the ability of ch128.1Av/b-SO6 and/or n(ch128.1Av/b-SO6) to kill tumor cells in 2F7-BR44 spheroids. Compared to the control, both ch128.1Av/b-SO6 and n(ch128.1Av/b-SO6) achieved comparable levels of cell killing in the outer layer of the 3D spheroids (FIGS. 4a-b). In contrast, with the improved penetration by n(ch128.1Av/b-SO6) within tumor spheroids, a significant enhancement of cell death in the core was seen with n(ch128.1Av/b-SO6) compared to ch128.1Av/b-SO6 (FIGS. 5a-b).

### Example 4—Safety in NSG Mice

### Methods

Male NOD.Cg-Prkdc scid Il2rg tm1Wjl/SzJ (NSG) mice aged week 6 were administered intravenously via retro-orbital venous sinus with 4 mg/kg of either ch128.1Av/b-SO6 or n(ch128.1Av/b-SO6) (n=3), with dosages normalized to the content of the ch128.1Av. Blood samples were collected daily during the first week, and subsequently on days 14 and 20. Plasma was extracted to assess alanine transaminase (ALT) levels via ELISA assays. Additionally, any inflammation at the eye mucosa injection sites was closely monitored through visual inspection.

### Results

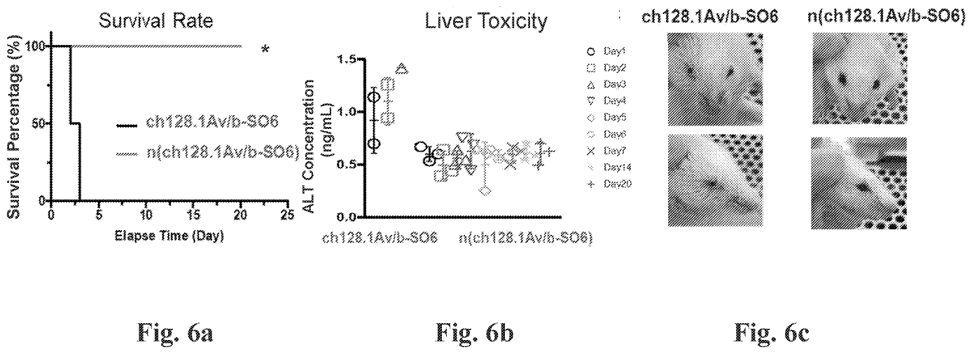

The proimmunotoxin n(ch128.1Av/b-SO6) can effectively shield the undesired toxicity of encapsulated immunotoxin. FIG. 6a shows that mice injected with ch128.1Av/b-SO6 showed systemic toxicity, necessitating euthanasia within 3 days. This toxicity is attributed non-TfR1 update of ch128.1Av/b-SO6 since ch128.1Av does not cross-react with mouse TfR1. In contrast, n(ch128.1Av/b-SO6) showed 100% survival rates, demonstrating significantly prolonged survival. Mice administered with ch128.1Av/b-SO6 have a significant increase in ALT levels, consistent with acute liver inflammation (FIG. 6b). In contrast, n(ch128.1Av/b-SO6) shows no impact on liver function. Moreover, n(ch128.1Av/b-SO6) does not cause acute inflammation at the injection sites around eyes (FIG. 6c).

### Example 5—In Vivo Efficacy in AIDS-NHL Xenograft Mouse Model with Brain Metastases

### Methods

The therapeutic efficacy of n(ch128.1Av/b-SO6) was evaluated using a xenograft AIDS-NHL model with 2F7-BR44 cells, which were genetically marked by both an mCherry fluorescent protein and a luciferase reporter. Male NSG mice aged 6 weeks were xenografted with 10⁶ 2F7-BR44 cells through tail vein to establish disseminated lymphoma with brain metastases. The mice were randomized into three groups and treated with PBS control, 0.1 mg/kg of ch128.1Av/b-SO6, or n(ch128.1Av/b-SO6) intravenously via tail vein on days 7 and 21 post-tumor xenograft (control n=3, treatment n=4). Longitudinal tumor progression in AIDS-NHL mice was monitored weekly using IVIS bioluminescent imaging (Lumina II IVIS, PerkinElmer) for four weeks. In vivo bioluminescence imaging was performed following subcutaneous injection of 4.5 mg D-luciferin (Pierce). Mice were imaged at the signal plateau (10 mins post-D-luciferin injection) under isoflurane anesthesia. The tumor burden was quantified as the total bioluminescent intensity (BLI) per second within a region of interest (whole body or head area) of the mouse using Living Image software (PerkinElmer); regions of interest were identically sized for all measurements. Mice were euthanized if they meet any of the following criteria: 1) reaching day 35 post-xenograft, 2) total BLI exceeding 10¹¹, or 3) experiencing a body weight loss exceeding 20%. Euthanasia is carried out in accordance with the approved animal protocol. Tumor-bearing organs, including the kidneys and bone marrow (BM), were harvested and processed for single-cell isolation using 40 μm cell strainers. Tumor burden in these organs was analyzed by measuring mCherry expression in 2F7-BR44 cells using the Attune NxT Flow Cytometer (ThermoFisher, Waltham, MA, USA). Concentrations of native ch128.1Av/b-SO6 and ch128.1Av/b-SO6 released from n(ch128.1Av/b-SO6) in tissue homogenates from the kidneys, brain, spleen, and liver were evaluated by ELISA against human IgG (Purified anti-human IgG Fc Antibody, BioLegend, San Diego, CA, USA).

### Results

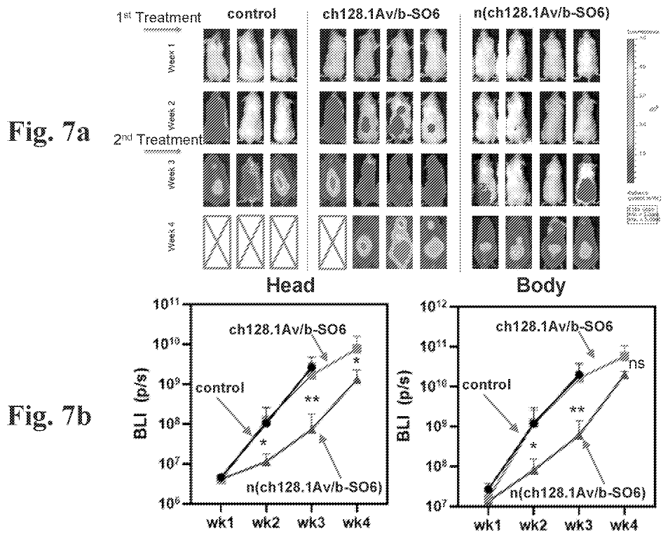

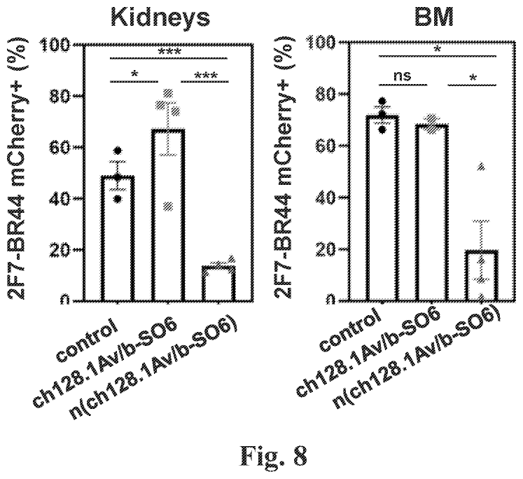

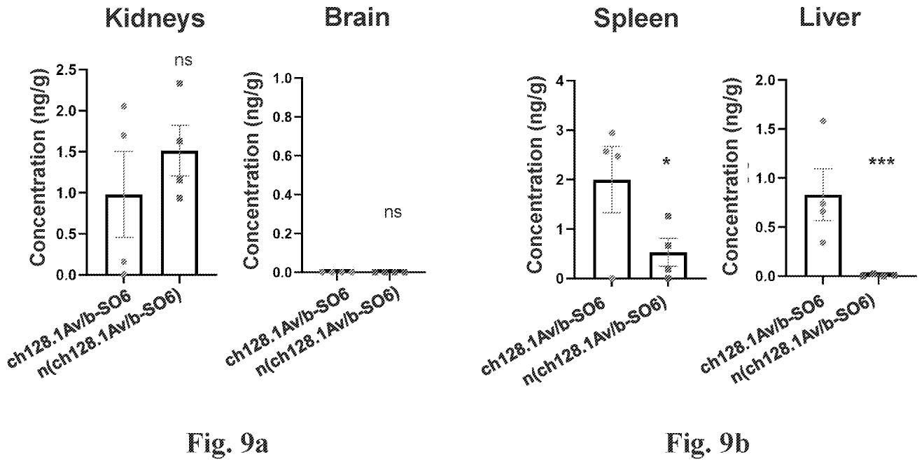

As shown in FIG. 7a, both ch128.1Av/b-SO6 and n(ch128.1Av/b-SO6) extend mouse lifespans by at least one week. Notably, treatment with n(ch128.1Av/b-SO6) significantly suppresses 2F7-BR44 progression and metastasis, as evidenced by a marked decrease in luciferase intensity from tumors in the head and throughout the entire body of the mice (FIG. 7b). Moreover, the tumor burden in two major metastatic organs, the kidneys and bone marrow (BM), is evaluated by flow cytometry. While treatment with ch128.1Av/b-SO6 had no effect on tumors in these organs, n(ch128.1Av/b-SO6) effectively reduced tumor formation in both, indicating enhanced antitumor efficacy through the design of proimmunotoxin nanodrugs (FIG. 8). Moreover, the accumulation of native ch128.1Av/b-SO6 and ch128.1Av/b-SO6 released from n(ch128.1Av/b-SO6) in various organs is compared using ELISA (FIGS. 9a-b). Mice treated with n(ch128.1Av/b-SO6) show improved accumulation of the released immunotoxin in the kidneys and reduced delivery to the spleen and liver, which are organs without tumor burden, thus underscoring the improved tumor targeting of the proimmunotoxin treatment.

### Example 6—In Vivo Efficacy with CXCL13 Conjugation in AIDS-NHL Xenograft Mouse Model with Brain Metastases

### Methods

The proimmunotoxin n(ch128.1Av/b-SO6) was further enhanced by conjugation with CXCL13. CXCL13 is a chemokine associated with B-cell NHL and interacted with CXCR5, a receptor expressed on B-cell NHL, to better target the tumors and further reduce non-tumor specific toxicity. n(ch128.1Av/b-SO6) reacted with CXCL13 ligands to synthesize n(ch128.1Av/b-SO6)^{CXCL13} through copper-free click chemistry. Dibenzocyclooctyne-S—S—N-hydroxysuccinimidyl ester (DBCO-SS-NHS) in DMSO (11 mg/ml) was added into n(ch128.1Av/b-SO6) solution at a 10:1 molar ratio. NHS-PEG4-Azide in DMSO (39 mg/ml) was added into CXCL13 solution at a 10:1 molar ratio. Both reactions were processed in an ice bath for 1 hour. Free reagents were removed by size-exclusion spin columns. The conjugation between DBCO-modified n(ch128.1Av/b-SO6) and azide-modified CXCL13 was reacted at 4° C. for 17 hours at a 1:1 molar ratio.

The therapeutic efficacy of n(ch128.1Av/b-SO6)^{CXCL13} was evaluated in a xenograft AIDS-NHL model. After being xenografted with 10⁶ 2F7-BR44 cells, mice were randomized into four groups and intravenously via tail vein treated with PBS control, 1 mg/kg of ch128.1Av/b-SO6, n(ch128.1Av/b-SO6), or n(ch128.1Av/b-SO6)^{CXCL13}, on days 7 and 21 post-xenograft (n=6). Longitudinal tumor progression in AIDS-NHL mice was monitored weekly using IVIS bioluminescent imaging (Lumina II IVIS, PerkinElmer) for four weeks. In vivo bioluminescence imaging was performed following subcutaneous injection of 4.5 mg D-luciferin (Pierce). Mice were imaged at the signal plateau (10 min post-D-luciferin injection) under isoflurane anesthesia. Mice were euthanized if they meet any of the following criteria: 1) reaching day 35 post-xenograft, 2) total BLI exceeding 10¹¹, or 3) experiencing a body weight loss exceeding 20%. Euthanasia is carried out in accordance with the approved animal protocol. Survival was based on the time from tumor challenge to mice reaching to euthanization criteria. Survival plots were generated using GraphPad Prism Version 9. Median survival and differences in survival (log-rank test) were determined using the same software.

### Results

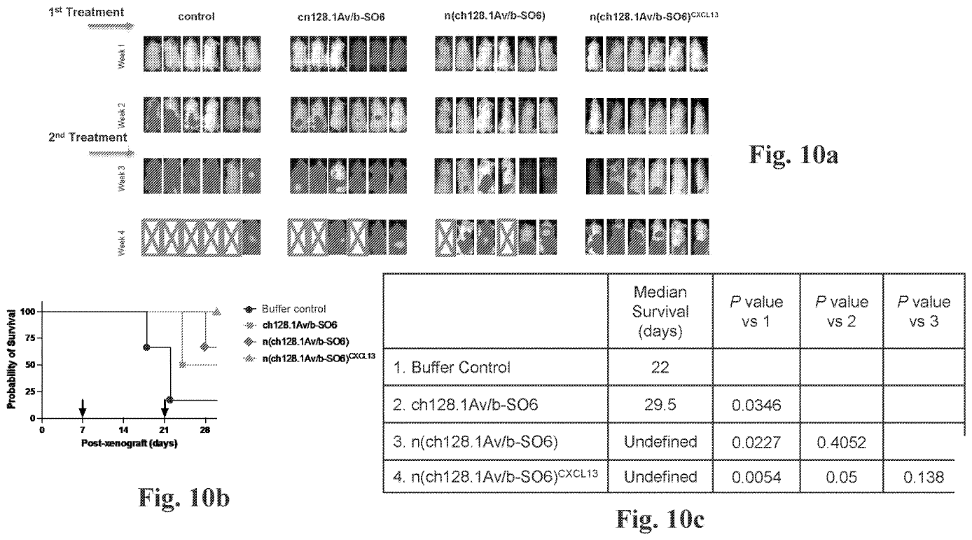

To assess the impact on NHL growth control, the effectiveness of ch128.1Av/b-SO6, n(ch128.1Av/b-SO6), and n(ch128.1Av/b-SO6)^{CXCL13} was evaluated in FIGS. 10a-b. All three treatments significantly suppressed tumor growth compared to buffer control. The imaging study is consistent with a stronger antitumor activity of n(ch128.1Av/b-SO6)^{CXCL13} compared to other groups. This imaging study also shows that both n(ch128.1Av/b-SO6) and n(ch128.1Av/b-SO6)^{CXCL13} suppressed lymphoma growth in the brain in at least half of the surviving mice at the end time point, consistent with the effective blood-brain barrier (BBB) penetration of these proimmunotoxin nanodrugs. Ultimately, treatment with n(ch128.1Av/b-SO6)^{CXCL13} extended the survival of AIDS-NHL mice by 1-2 weeks compared to treatment with ch128.1Av/b-SO6.

### REFERENCES

The following references, and those cited herein, to the extent that they provide exemplary procedural or other details supplementary to those set forth herein, are specifically incorporated herein by reference.
1 Siegel, R. L., Miller, K. D., Wagle, N. S. & Jemal, A. Cancer statistics, 2023. CA Cancer J Clin 73, 17-48, (2023).
2 Gibson, T. M., Morton, L. M., Shiels, M. S., Clarke, C. A. & Engels, E. A. Risk of non-Hodgkin lymphoma subtypes in HIV-infected people during the HAART era: a population-based study. AIDS 28, 2313-2318, (2014).
3 Kimani, S. M., Painschab, M. S., Horner, M. J., Muchengeti, M., Fedoriw, Y., Shiels, M. S. & Gopal, S. Epidemiology of haematological malignancies in people living with HIV. Lancet HIV 7, e641-e651, (2020).
4 Nishijima, T., Inaba, Y., Kawasaki, Y., Tsukada, K., Teruya, K., Kikuchi, Y., Gatanaga, H. & Oka, S. Mortality and causes of death in people living with HIV in the era of combination antiretroviral therapy compared with the general population in Japan. AIDS 34, 913-921, (2020).
5 Bonnet, F., Balestre, E., Thiebaut, R., Morlat, P., Pellegrin, J. L., Neau, D. & Dabis, F. (Groupe d′Epidemiologie Clinique du SIDA en Aquitaine). Factors associated with the occurrence of AIDS-related non-Hodgkin lymphoma in the era of highly active antiretroviral therapy: Aquitaine Cohort, France. Clin Infect Dis 42, 411-417, (2006).
6 Carbone, A., Gloghini, A., Capello, D. & Gaidano, G. Genetic pathways and 6 histogenetic models of AIDS-related lymphomas. Eur J Cancer 37, 1270-1275, (2001).
7 Barta, S. K., Joshi, J., Mounier, N., Xue, X., Wang, D., Ribera, J. M., Navarro, J. T., Hoffmann, C., Dunleavy, K., Little, R. F., Wilson, W. H., Spina, M., Galicier, L., Noy, A. & Sparano, J. A. Central nervous system involvement in AIDS-related lymphomas. Br J Haematol 173, 857-866, (2016).
8 Bohlius, J., Schmidlin, K., Costagliola, D., Fatkenheuer, G., May, M., Caro Murillo, A. M., Mocroft, A., Bonnet, F., Clifford, G., Touloumi, G., Miro, J. M., Chene, G., Lundgren, J. & Egger, M. (Collaboration of Observational, H. I. V. Epidemiological Research Europe study group). Prognosis of HIV-associated non-Hodgkin lymphoma in patients starting combination antiretroviral therapy. AIDS 23, 2029-2037, (2009).
9 Essaghir, A. & Demoulin, J. B. A minimal connected network of transcription factors regulated in human tumors and its application to the quest for universal cancer biomarkers. PLOS One 7, e39666, (2012).
10 Daniels, T. R., Delgado, T., Rodriguez, J. A., Helguera, G. & Penichet, M. L. The transferrin receptor part I: Biology and targeting with cytotoxic antibodies for the treatment of cancer. Clin Immunol 121, 144-158, (2006).
11 Daniels-Wells, T. R. & Penichet, M. L. Transferrin receptor 1: a target for antibody-mediated cancer therapy. Immunotherapy 8, 991-994, (2016).
12 Shen, Y., Li, X., Dong, D., Zhang, B., Xue, Y. & Shang, P. Transferrin receptor 1 in cancer: a new sight for cancer therapy. Am J Cancer Res 8, 916-931, (2018).
13 Lelievre, P., Sancey, L., Coll, J. L., Deniaud, A. & Busser, B. Iron Dysregulation in Human Cancer: Altered Metabolism, Biomarkers for Diagnosis, Prognosis,
Monitoring and Rationale for Therapy. Cancers (Basel) 12, (2020).
14 Candelaria, P. V., Leoh, L. S., Penichet, M. L. & Daniels-Wells, T. R. Antibodies Targeting the Transferrin Receptor 1 (TfR1) as Direct Anti-cancer Agents. Front Immunol 12, 607692, (2021).
15 Rhodes, D. R., Kalyana-Sundaram, S., Mahavisno, V., Varambally, R., Yu, J., Briggs, B. B., Barrette, T. R., Anstet, M. J., Kincead-Beal, C., Kulkarni, P., Varambally, S., Ghosh, D. & Chinnaiyan, A. M. Oncomine 3.0: genes, pathways, and networks in a collection of 18,000 cancer gene expression profiles. Neoplasia (New York, N.Y.) 9, 166-180, (2007).
16 Uhlén, M., Fagerberg, L., Hallström, B. M., Lindskog, C., Oksvold, P., Mardinoglu, A., Sivertsson, A., Kampf, C., Sjöstedt, E., Asplund, A., Olsson, I., Edlund, K., Lundberg, E., Navani, S., Szigyarto, C. A.-K., Odeberg, J., Djureinovic, D., Takanen, J. O., Hober, S., Alm, T., Edqvist, P.-H., Berling, H., Tegel, H., Mulder, J., Rockberg, J., Nilsson, P., Schwenk, J. M., Hamsten, M., von Feilitzen, K. & Forsberg, M. et al. Proteomics. Tissue-based map of the human proteome. Science (New York, N.Y.) 347, 1260419-1260419, (2015).
17 Daniels, T. R., Delgado, T., Helguera, G. & Penichet, M. L. The transferrin receptor part II: targeted delivery of therapeutic agents into cancer cells. Clin Immunol 121, 159-176, (2006).
18 Daniels, T. R., Bernabeu, E., Rodriguez, J. A., Patel, S., Kozman, M., Chiappetta, D. A., Holler, E., Ljubimova, J. Y., Helguera, G. & Penichet, M. L. The transferrin receptor and the targeted delivery of therapeutic agents against cancer. Biochim Biophys Acta 1820, 291-317, (2012).
19 Ng, P. P., Dela Cruz, J. S., Sorour, D. N., Stinebaugh, J. M., Shin, S. U., Shin, D. S., Morrison, S. L. & Penichet, M. L. An anti-transferrin receptor-avidin fusion protein exhibits both strong proapoptotic activity and the ability to deliver various molecules into cancer cells. Proc Natl Acad Sci USA 99, 10706-10711, (2002).
20 Daniels, T. R., Ng, P. P., Delgado, T., Lynch, M. R., Schiller, G., Helguera, G. & Penichet, M. L. Conjugation of an anti-transferrin receptor IgG3-avidin fusion protein with biotinylated saporin results in significant enhancement of its cytotoxicity against malignant hematopoietic cells. Mol Cancer Ther 6, 2995-3008, (2007).
21 Daniels-Wells, T. R., Helguera, G., Rodriguez, J. A., Leoh, L. S., Erb, M. A., Diamante, G., Casero, D., Pellegrini, M., Martinez-Maza, O. & Penichet, M. L. Insights into the mechanism of cell death induced by saporin delivered into cancer cells by an antibody fusion protein targeting the transferrin receptor 1. Toxicol In Vitro 27, 220-231, (2013).
22 Wu, D., Qin, M., Xu, D., Wang, L., Liu, C. Y., Ren, J., Zhou, G., Chen, C., Yang, F. M., Li, Y. Y., Zhao, Y., Huang, R. Y., Pourtaheri, S., Kang, C. S., Kamata, M., Chen, I. S. Y., He, Z. L., Wen, J., Chen, W. & Lu, Y. F. A Bioinspired Platform for Effective Delivery of Protein Therapeutics to the Central Nervous System. Advanced Materials 31, (2019).
23 Wen, J., Wu, D., Qin, M., Liu, C., Wang, L., Xu, D., Vinters, H. V., Liu, Y., Kranz, E., Guan, X., Sun, G., Sun, X., Lee, Y., Martinez-Maza, O., Widney, D., Lu, Y., Chen, I. S. Y. & Kamata, M. Sustained delivery and molecular targeting of a therapeutic monoclonal antibody to metastases in the central nervous system of mice. Nat Biomed Eng 3, 706-716, (2019).
24 Han, L., Liu, C., Qi, H., Zhou, J., Wen, J., Wu, D., Xu, D., Qin, M., Ren, J., Wang, Q., Long, L., Liu, Y., Chen, I., Yuan, X., Lu, Y. & Kang, C. Systemic Delivery of Monoclonal Antibodies to the Central Nervous System for Brain Tumor Therapy. Adv Mater 31, e1805697, (2019).
25 Wen, J., Anderson, S. M., Du, J., Yan, M., Wang, J., Shen, M., Lu, Y. & Segura, T. Controlled protein delivery based on enzyme-responsive nanocapsules. Adv Mater 23, 4549-4553, (2011).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The publications listed in the application, to the extent that they provide exemplary procedural or other details supplementary to those set forth herein, are specifically incorporated herein by reference.

What is claimed is:

1. A nanodrug comprising a toxin encapsulated by a polymer shell.

2. The nanodrug of claim 1, wherein the toxin comprises an immunotoxin.

3. The nanodrug of claim 2, wherein the immunotoxin comprises an anti-TfR1 antibody.

4. The nanodrug of claim 3, wherein the immunotoxin comprises a human antibody, a humanized antibody, a mouse antibody, and/or a chimeric antibody.

5. The nanodrug of claim 4, wherein the chimeric antibody comprises a mouse/human chimeric antibody.

6. The nanodrug of claim 3, wherein the anti-TfR1 antibody comprises ch128.1 and/or hu128.1.

7. The nanodrug of claim 2, wherein the immunotoxin comprises a functional fragment of an anti-TfR1 antibody.

8. The nanodrug of claim 2, wherein the immunotoxin comprises a Fab fragment of an anti-TfR1 antibody.

9. The nanodrug of claim 2, wherein the immunotoxin comprises complementarity-determining regions (CDRs) of an anti-TfR1 antibody.

10. The nanodrug of claim 2, wherein the immunotoxin comprises a single chain variable fragment (scFv) of an anti-TfR1 antibody.

11. The nanodrug of claim 2, wherein the immunotoxin comprises a peptide and/or aptamer that binds TfR1.

12. The nanodrug of any one of claims 2-7, wherein the immunotoxin comprises a ribosome-inactivating protein (RIP).

13. The nanodrug of claim 12, wherein the ribosome-inactivating protein comprises saporin.

14. The nanodrug of any one of claims 2-7, wherein the immunotoxin comprises saporin, diphtheria toxin, Pseudomonas aeruginosa exotoxin A, ricin, monomethyl auristatin E (MMAE), or any combination thereof.

15. The nanodrug of any one of claims 2-10, wherein the immunotoxin comprises an antibody-avidin fusion protein and/or antibody-streptavidin fusion protein.

16. The nanodrug of any one of claims 2-11, wherein the immunotoxin comprises a biotinylated saporin.

17. The nanodrug of any one of claims 1-16, wherein the polymer shell comprises 2-methacryloyloxyethyl phosphorylcholine.

18. The nanodrug of any one of claims 1-17, wherein the polymer shell comprises a degradable peptide crosslinker.

19. The nanodrug of any one of claims 1-18, wherein the polymer shell comprises polymer capable of being degraded by a matrix metalloproteinase (MMP), urokinase-type plasminogen activator (uPA), membrane type 1 serine protease (MT-SP1/matriptase), legumain, or any combination thereof.

20. The nanodrug of claim 19, wherein the MMP comprises MMP-2 and/or MMP-14.

21. The nanodrug of any one of claims 1-20, wherein the polymer shell comprises targeting molecules.

22. The nanodrug of claim 21, wherein the targeting molecules comprise antitumor antigen antibodies

23. The nanodrug of claim 21 or 22, wherein the targeting molecules comprise molecules capable of targeting central nervous tissue.

24. The nanodrug of claim 23, wherein the central nervous tissue comprises brain tissue.

25. The nanodrug of any one of claims 21-24, wherein the targeting molecules comprise blood-brain barrier (BBB) ligands.

26. The nanodrug of any one of claims 21-25, wherein the targeting molecules comprise a choline analog.

27. The nanodrug of claim 26, wherein the choline analog is methacryloyloxyethyl phosphorylcholine (MPC).

28. The nanodrug of any one of claims 21-25, wherein the tumor targeting molecule comprises a chemokine.

29. The nanodrug of claim 28, wherein the chemokine is CXCL13.

30. A method of treating cancer in a patient, the method comprising administering the nanodrug of any one of claims 1-29 to the patient.

31. The method of claim 30, wherein the cancer is a brain cancer.

32. The method of claim 30 or 31, wherein the cancer comprises a brain tumor.

33. The method of any one of claims 30-26, wherein the cancer comprises cancerous cells in brain tissue of the patient.

34. The method of any one of claims 30-33, wherein the cancer comprises cancerous cells in a deep tissue of the patient.

35. The method of any one of claims 30-34, wherein the cancer is a blood cancer.

36. The method of any one of claims 30-35, wherein the cancer is a lymphoma.

37. The method of any one of claims 30-36, wherein the cancer is non-Hodgkin lymphoma (NHL).

38. The method of any one of claims 30-34, wherein the cancer is a solid tumor.

39. The method of any one of claims 30-38, wherein the cancer is metastatic.

40. The method of any one of claims 30-39, wherein the cancer is a primary cancer.

41. The method of any one of claims 30-40, further comprising administering an additional therapeutic composition to the individual.

42. The method of any one of claims 30-41, wherein the administering is done subcutaneously, intravenously, intraperitoneally, intratumorally, or intrathecally.

43. A method of delivering a toxin to a tissue in a patient comprising, the method comprising administering the nanodrug of claims 1-29.

44. The method of claim 43, wherein the cancer is a brain cancer.

45. The method of claim 43 or 44, wherein the cancer comprises a brain tumor.

46. The method of any one of claims 43-39, wherein the cancer comprises cancerous cells in brain tissue of the patient.

47. The method of any one of claims 43-46, wherein the cancer comprises cancerous cells in a deep tissue of the patient.

48. The method of any one of claims 43-47, wherein the cancer is a blood cancer.

49. The method of any one of claims 43-48, wherein the cancer is a lymphoma.

50. The method of any one of claims 43-49, wherein the cancer is NHL.

51. The method of any one of claims 43-47, wherein the cancer is a solid tumor.

52. The method of any one of claims 43-51, wherein the cancer is metastatic.

53. The method of any one of claims 43-52, wherein the cancer is a primary cancer.

54. The method of any one of claims 43-50, further comprising administering an additional therapeutic composition to the individual.

55. The method of any one of claims 43-54, wherein the administering is done subcutaneously, intravenously, intraperitoneally, intratumorally, or intrathecally.
